(19) United States
(12) Patent Application Publication
(10) Pub. No.: US 2004/0199004 A1
(43) Pub. Date: Oct. 7, 2004

(54) Novel mesogens

(75) Inventors: Stephen T. Wellinghoff, San Antonio, TX (US); Douglas P. Hanson, San Antonio, TX (US)

Correspondence Address: PAULA D. MORRIS & ASSOCIATES, P.C., d/b/a THE MORRIS LAW FIRM, P.C., 10260 WESTHEIMER, SUITE 360, HOUSTON, TX, 77042-3110, US

(73) Assignee: Southwest Research Institute, San Antonio, TX

(21) Appl. No.: 10/811,090

(22) Filed: Mar. 26, 2004

Related U.S. Application Data
(63) Continuation-in-part of application No. 10/056,121, filed on Jan. 23, 2002.
(60) Provisional application No. 60/263,387, filed on Jan. 23, 2001.
(60) Provisional application No. 60/263,392, filed on Jan. 23, 2001.
(60) Provisional application No. 60/263,388, filed on Jan. 23, 2001.

(51) Int. Cl. C07C 69/76

(52) U.S. Cl. 560/19; 560/66

(57) ABSTRACT

Mesogens having the following general formula:

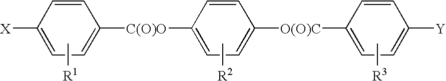

wherein

X and Y independently are selected from the group consisting of amino groups, polymerizable groups, and groups having the following general structure:

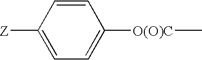

wherein Z is selected from the group consisting of terminal functionalities and polymerizable groups; provided that when X is a polymerizable group, Y is an amino group;

R² is a bulky organic group having a bulk greater than R¹ and R³ whereby, when both X and Y are polymerizable groups, said bulk is adapted to provide sufficient steric hindrance to achieve a nematic state at room temperature while suppressing crystallinity at room temperature, thereby providing effective rheology and workability at room temperature; and

R¹ and R³ are selected from groups less bulky than R² adapted to maintain said nematic state.

### PRIORITY DATA

The present application is a continuation-in-part of application Ser. No. 10/056,121 filed Jan. 23, 2002, pending, incorporated herein by reference, which claims the benefit of the following provisional applications, all filed Jan.23, 2001: Ser. No. 60/263,387; Ser. No. 60/263,392; Ser. No. 60/263,388.

### GOVERNMENT RIGHTS CLAUSE

The U.S. government has certain rights in this invention pursuant to grant number NIDCR 1 P01 DE11688.

### FIELD OF THE APPLICATION

The invention relates to novel mesogens.

### BACKGROUND

Photocurable resins which are transparent or translucent, radioopaque, have good workability, and have good mechanical strength and stability are useful in medical, dental, adhesive, and stereolithographic applications.

Low polymerization shrinkage is an important property for such resins. In dental applications, the phrase “zero polymerization shrinkage” typically means that the stresses accumulated during curing do not debond the dentin-restorative interface or fracture the tooth or restorative, which can result in marginal leakage and microbial attack of the tooth. Low polymerization shrinkage also is important to achieve accurate reproduction of photolithographic imprints and in producing optical elements.

Another advantageous property for such resins is maintenance of a liquid crystalline state during processing. For comfort in dental applications, the resin should be curable at “room temperature,” defined herein as typical ambient temperatures up to body temperature. Preferred curing temperatures are from about 20° C. to about 37° C. Mesogens which have been found to polymerize in a relatively stable manner at such temperatures are bis 1,4[4′-(6′-methacryloxyhexyloxy) benzoyloxy] t-butylphenylene mesogens and their structural derivatives. These mesogens have the following general structure:

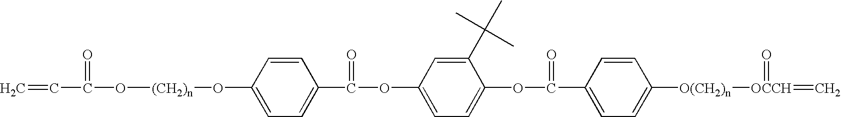

Known synthetic methods for producing these mesogens are costly and have relatively low yields. New synthetic methods and new methods and compositions for controlling rheology of these photocurable resins are needed.

### SUMMARY

Mesogens are provided having the following general formula:

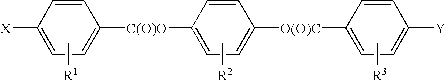

wherein

X and Y independently are selected from the group consisting of amino groups, polymerizable groups, and groups having the following general structure:

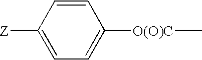

wherein Z is selected from the group consisting of a terminal functionality and a polymerizable group; provided that when X is a polymerizable group, Y is an amino group;

R² is a bulky organic group having a bulk greater than R¹ and R³ whereby, when both X and Y are polymerizable groups, said bulk is adapted to provide sufficient steric hindrance to achieve a nematic state at room temperature while suppressing crystallinity at room temperature, thereby providing effective rheology and workability at room temperature; and

R¹ and R³ are selected from groups less bulky than R² adapted to maintain said nematic state.

### DETAILED DESCRIPTION

The application provides novel having the following general formula:

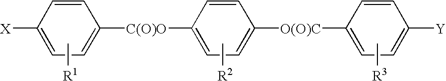

wherein

X and Y independently are selected from the group consisting of amino groups, polymerizable groups, and groups having the following general structure:

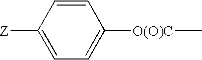

wherein Z is selected from the group consisting of a terminal functionality and a polymerizable group; provided that when X is a polymerizable group, Y is an amino group;

R² is a bulky organic group having a bulk greater than R¹ and R³ whereby, when both X and Y are polymerizable groups, said bulk is adapted to provide sufficient steric hindrance to achieve a nematic state at room temperature while suppressing crystallinity at room temperature, thereby providing effective rheology and workability at room temperature; and

R¹ and R³ are selected from groups less bulky than R² adapted to maintain said nematic state.

Suitable R² groups generate asymmetry in the packing of the molecules, and include, but are not necessarily limited to alkyl groups having from about 1 to 6 carbon atoms and aryl groups. Preferred R² groups include, but are not necessarily limited to alkyl groups having from about 1 to about 4 carbon atoms and phenyl groups. More preferred R² groups are methyl groups, t-butyl groups, isopropyl groups, secondary butyl groups, and phenyl groups. Most preferred R² groups are methyl groups and t-butyl groups. Preferred R¹ and R³ groups are selected from the group consisting of hydrogen atoms and methyl groups, depending upon the relative bulk of R¹, R³, and R².

“Terminal functionalities” are defined as protective groups and precursors to polymerizable groups, which generally comprise functionalities that readily react with “polymerizable groups” to form reactive ends. Suitable terminal functionalities independently are selected from the group consisting of hydroxyl groups, amino groups, sulfhydryl groups, halogen atoms, “spacer groups”, and groups comprising “spacer groups,” particularly spacer groups comprising a terminal functionalities selected from the group consisting of hydroxyl groups, amino groups, sulfhydryl groups, and halogen atoms. “Spacer groups” are defined herein as selected from the group consisting of H—(CH₂)ₙ—O— groups, Cl(CH₂)ₙ—O— groups, Br(CH₂)ₙ—O— groups, I(CH₂)ₙ—O—, wherein n is from about 2 to about 12, preferably from about 2 to about 9, more preferably from about 2 to about 6, and most preferably 6, and the CH₂ groups independently can be substituted by oxygen, sulfur, or an ester group; provided that at least 2 carbon atoms separate said oxygen or said ester group.

Where the mesogen is a polymerizable mesogen, X and/or Y are “polymerizable groups,” defined as groups that may be polymerized by nucleophilic addition, free radical polymerization, or a combination thereof. Preferred polymerizable groups are polymerizable by Michael addition. Michael addition requires the addition of a nucleophile and an electron deficient alkene. Groups suitable for polymerization by Michael addition include but are not necessarily limited to the examples found in A. Michael, J. Prakt. Chem. [2] 35, 349 (1887); R. Connor and W. R. McClelland, J. Org. Chem., 3, 570 (1938); and C. R. Hauser, M. T. Tetenbaum, J. Org. Chem., 23, 1146 (1959), all of which are incorporated by reference herein.

Examples of suitable polymerizable groups include, but are not necessarily limited to substituted and unsubstituted alkenyl ester groups comprising a polymerizable unsaturated carbon-carbon bond, wherein said alkenyl group has from about 2 to about 12 carbon atoms, preferably from about 2 to about 9 carbon atoms, more preferably from about 2 to about 6 carbon atoms. In one embodiment, said substituted alkenyl ester groups comprise at least one halogen atom selected from the group consisting of chorine atoms, bromine atoms, and iodine atoms. Preferred alkenyl esters are acryloyloxy alkoxy groups and methacryloyloxy alkoxy groups. More preferred polymerizable groups include, but are not necessarily limited to cinnamoyloxy groups, acryloyloxy groups, methacryloxy groups, as well as thioalkyloxy groups, acryloyloxy alkoxy groups, and methacryloyloxy alkoxy groups comprising an alkyl moiety having from about 2 to about 12 carbon atoms, preferably about 6 carbon atoms, said alkyl moieties comprising CH₂ groups which independently can be substituted by oxygen, sulfur, or an ester group; provided that at least 2 carbon atoms separate said oxygen or said ester group. Because asymmetry suppresses crystallinity while maintaining a nematic state, it is preferred for X and Y to be different groups.

Mesomers of higher temperature nematic stability are “mesogenic dimers,” formed by reacting X or Y on separate mesogen molecules with opposite ends of a bridging agent. Examples of suitable bridging agents include, but are not necessarily limited to dicarboxylic acids (preferably α,ω-carboxylic acids) having from about 4 to about 12 carbon atoms, preferably from about 6 to about 10 carbon atoms, and oligodialkylsiloxanes preferably comprising alkyl groups having from about 1 to about 3 carbon atoms, most preferably methyl groups. When the mesomer is a dimer or another structure comprising components that inherently interfere with crystallization at room temperature, R² need not have a bulk greater than R¹ and R³. Hence, R² may be hydrogen, a halogen atom, or another substituent having lesser bulk than a methylene group.

Depending on the sample preparation, the volumetric photopolymerization shrinkage of these materials at room temperature varies from about 0.9 to about 1.7%, which is a factor of 6-4X improvement over commercially available blends containing 2,2-bis[p-(2′-hydroxy-3′-methacryloxypropoxy)phenylene] propane (“bis-GMA”). Preferably, the polymerizable mesogens exhibit “low polymerization shrinkage,” defined herein as about 3 vol. % change or less, preferably about 2 vol. % change or less.

Dimers

The following is a ChemSketch 4 rendition of the minimum energy conformation of {C0[H,TB,H] (MeAcry)(O)}₂(seb). As expected the most stable conformation is an extended form with a very high molecular length to width ratio which is likely to form high T_{n->I} liquid crystal monomers.

[image]

A minimum energy conformation of a preferred mesogenic dimer is decanedioic acid bis-(4-{2-tert-butyl-4-[4-(2-methyl-acryloyloxy)-benzoyloxy]-phenoxycarbonyl}-phenyl) ester {C0[H,TB,H] (MeAcry)(O)}₂(seb):

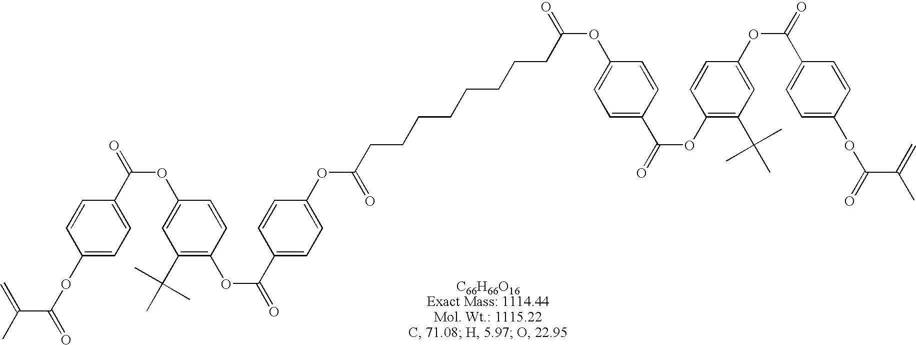

An exemplary novel mesogen is alkylenedioic bis-(4-{2-alkyl-4-[4-(hydroxy)-benzoyloxy]-phenoxycarbonyl}-phenyl) ester having the following general structure:

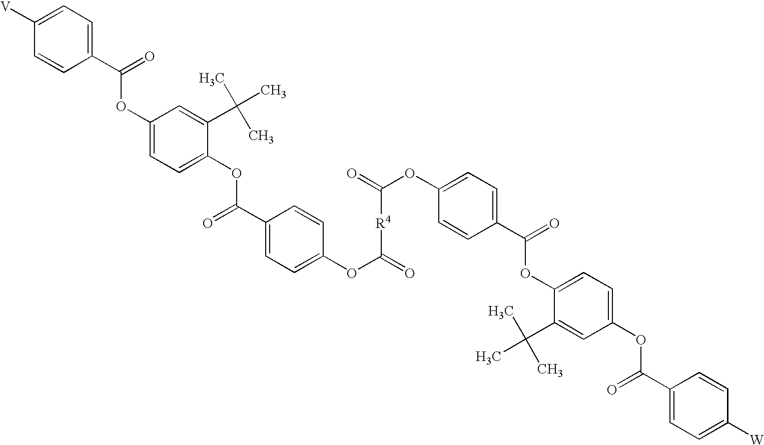

wherein

R⁴ has from about 2 to about 20 carbon atoms, preferably from about 2 to about 12 carbon atoms, and most preferably from about 6 to about 12 carbon atoms.

the alkyl substituent on the central aromatic group of the aromatic ends includes, but is not necessarily limited to t-butyl groups, isopropyl groups, and secondary butyl groups. Most preferred are t-butyl groups; and,

V and W are selected from the group consisting of terminal functionalities and polymerizable groups. In platform molecules, V and W are terminal functionalities. In polymerizable mesogens, V and/or W are polymerizable groups.

Additional exemplary mesogens have the following general structure:

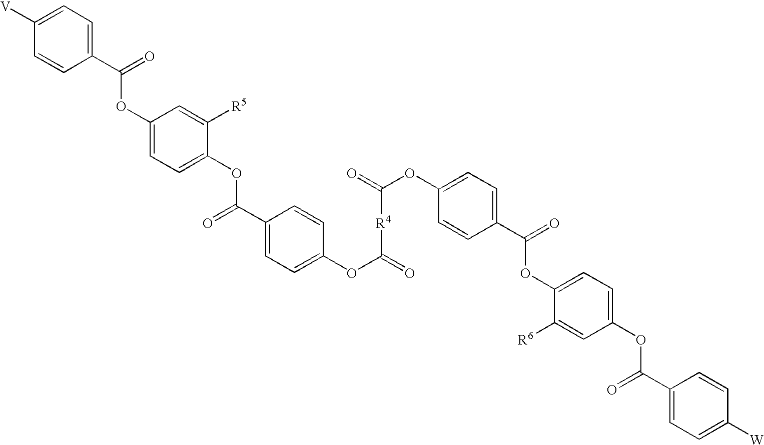

wherein

R⁵ and R⁶ are selected from the group consisting of hydrogen, halogen, n-alkyl groups having from about 1 to 6 carbon atoms, aryl groups, and bulky organic groups; and,

V and W independently are selected from the groups comprising polymerizable groups and terminal functionalities.

Suitable terminal functionalities independently are selected from the group consisting of hydroxyl groups, amino groups, and sulfhydryl groups. Most preferred terminal functionalities are hydroxyl groups.

Suitable polymerizable groups may be polymerized by nucleophilic addition, free radical polymerization, or a combination thereof. Preferred polymerizable groups are polymerizable by Michael addition. Michael addition requires the addition of a nucleophile and an electron deficient alkene. Groups suitable for polymerization by Michael addition include but are not necessarily limited to the examples found in A. Michael, J. Prakt. Chem. [2] 35, 349 (1887); R. Connor and W. R. McClelland, J. Org Chem., 3, 570 (1938); and C. R. Hauser, M. T. Tetenbaum, J. Org Chem., 23, 1146 (1959), all of which are incorporated by reference herein.

Examples of suitable polymerizable groups include, but are not necessarily limited to substituted and unsubstituted alkenyl ester groups comprising a polymerizable unsaturated carbon-carbon bond, wherein said alkenyl group has from about 2 to about 12 carbon atoms, preferably from about 2 to about 9 carbon atoms, more preferably from about 2 to about 6 carbon atoms. Preferred alkenyl esters are acryloyl alkoxy groups, methacryloyloxy alkoxy groups, acryloyloxy groups, and methacryloyloxy groups. V and W may be the same or different, depending upon the application. In a preferred application—a dental application—V and W comprise terminal alkenyl groups.

These alkylenedioic bis-(4-{2-alkyl-4-[4-(hydroxy)-benzoyloxy]-phenoxycarbonyl}-phenyl) esters are novel compounds, and may be used as “platform molecules,” or polymerizable mesogens. A most preferred alkylenedioic bis-(4-{2-alkyl-4-[4-(hydroxy)-benzoyloxy]-phenoxycarbonyl}-phenyl) ester is decanedioic acid bis-(4-{2-tert-butyl-4-[4-(hydroxy)-benzoyloxy] -phenoxycarbonyl}-phenyl) ester.

Low Polymerization Shrinkage

The mesogens exhibit “low polymerization shrinkage” preferably no more than 3 vol. % change, more preferably no more than about 2 volume percent change. Polymerization shrinkage is measured by codissolving the monomers in dichloromethane with 0.3 wt. % camphorquinone photoinitiator, 100 ppm benzoquinone and 1 wt. % N,N′ dimethylaminoethyl methacrylate activator and subsequently pumping off the solvent, all under yellow light. The monomers are then polymerized in film or droplet form in less than 1 minute by exposure to a dental curing light (Dentsply Spectrum Curing Lamp) with a significant output at 420 nm.

FTIR spectroscopy (Nicolet Magna-IR 560) is used to measure the degree of cure by observing the decrease in the 1637 cm⁻¹ alkene band vs. the aromatic internal thickness band at 1603 cm⁻¹. Thin film measurements that avoid oxygen inhibition are performed by sandwiching the monomer between polyvinylidene chloride films, which have an optical window in the wavelength region of interest. The IR spectrum of solid droplets is evaluated using a single bounce reflectance measurement. The flat bottom surface of the droplet is pressed against the germanium lens of a Spectra Tech Thunderdome attachment.

Polymerization of the monomers can be observed between transparent polyvinylidene chloride films under cross-polarized optical microscopy in the heated stage of a Nikon Optimat microscope. Little change in the local birefringence and thus local orientation is noted upon polymerization at room temperature or upon heating to 180° C.

Fracture Toughness

Compact tension samples (ASTM E399) with known edge crack length are fabricated by photocuring the monomer with initiator and activator in silicone molds. After polishing the surface with 600 grit polishing agent and soaking in physiologic saline at 37° C. for 24 hours the samples were tested at room temperature under displacement control at 1 mm/min until failure.

The fracture toughness of the crosslinked, amorphous glass suitable is about 0.4 MPa-m^{1/2}, preferably about 0.5 MPa-m^{1/3}, which is identical to that found for photocured, isotropic dimethacrylate based resins such as GTE resin.

Fillers

Considerable amounts of soluble impurity can be added to the polymerizable mesogens, or a mixture comprising the polymerizable mesogens, without changing the T_{nematic->isotropic} transition temperature of the polymerizable mesogens. Thus, a high volume fraction of filler can be added to the polymerizable mesogens and still form a composite that maintains desirable, low viscosity flow and low polymerization shrinkage characteristics at temperatures of curing. Commercial products add up to about 70-80 wt % filler. A preferred embodiment uses about 30 wt. % filler.

A variety of fillers may be used, including but not necessarily limited to silica fillers, glass fillers, and amphoteric nano-sized metal oxide particles. Preferred fillers are amphoteric nano-sized metal oxide particles having a diameter in nanometers which is sufficiently small to provide transparency effective for photopolymerization but sufficiently large to provide effective fracture toughness after photopolymerization. Substantially any “metal” capable of forming an amphoteric metal oxide may be used to form the metal oxide particles. Suitable metallic elements include, but are not necessarily limited to niobium, indium, titanium, zinc, zirconium, tin, cerium, hafnium, tantalum, tungsten, and bismuth. Also suitable in place of the metal in the oxide is the semi-metallic compound, silicon. As used herein, unless otherwise indicated, the term “metal oxide” is defined to include silicon, and the word “metal,” when used to refer to the metal oxide is intended to also refer to silicon.

The metal oxides may be made of a single metal, or may be a combination of metals, alone or combined with other impurities or “alloying” elements, including, but not necessarily limited to aluminum, phosphorus, gallium, germanium, barium, strontium, yttrium, antimony, and cesium.

A monomeric liquid crystal (LC) containing a high volume fraction of filler nanoparticles is a highly constrained system. As a result, at least for some monomeric species, both smectic and crystalline transitions should be suppressed. The consequent widening of the stability range of nematic mesophase should permit the composite to polymerize at much lower temperatures than in unfilled systems, resulting in lower polymerization shrinkage.

The metal oxide nanoparticles may be prepared using any known methods, such as “sol-gel” techniques, direct hydrolysis of metal alkoxides by water addition, forced hydrolysis of relatively low-cost metal salts, or non-hydrolytic reactions of metal alkoxides with metal halide salts. Examples of such procedures are shown in the following references, each of which is incorporated herein by reference: W. Stöber and A. Fink, J. of Colloid and Interface Science, v. 26, 62-69 (1968); M. Z.-C. Hu, M. T. Harris, and C. H. Byers, J. of Colloid and Interface Science, v. 198, 87-99 (1988); M. Ocaña and E. Matijević, J. of Materials Research, v. 5(5), 1083-1091 (1990); L. Lerot, F. LeGrand, P. de Bruycker, J. of Materials Science, v. 26, 2353-2358 (1991); H. Kumazawa, Y. Hori, and E. Sada, The Chemical Eng'g. Journal, v. 51, 129-133 (1993); S. K. Saha and P. Pramanik, J. of Non-Crystalline Solids, v. 159, 31-37 (1993); M. Andrianainarivelo, R. Corriu, D. Leclercq, P. H. Mutin, and A. Vioux, J. of Materials Chemistry, v. 6(10), 1665-1671 (1996); F. Garbassi, L. Balducci, R. Ungarelli, J. of Non-Crystalline Solids, v. 223, 190-199 (1998); J. Spatz, S. Mössmer, M. Mo[umlaut]ller, M. Kocher, D. Neher, and G. Wegner, Advanced Materials, v. 10(6), 473-475 (1998); R. F. de Farias, and C. Airoldi, J. of Colloid and Interface Science, v. 220, 255-259 (1999); T. J. Trentler, T. E. Denler, J. F. Bertone, A. Agrawal, and V. L. Colvin, J. of the Am. Chemical Soc., v. 121, 1613-1614 (1999); Z. Zhan and H. C. Zheng, J. of Non-Crystalline Solids, v. 243, 26-38 (1999); M. Lade, H. Mays, J. Schmidt, R. Willumeit, and R. Schomäcker, Colloids and Surfaces A: Physiochemical and Eng'g Aspects, v. 163, 3-15 (2000); and the procedure described in “Sol-gel processing with inorganic metal salt precursors,” authored by “Michael” Zhong Cheng Hu, licensable via Oak Ridge National Laboratory under ORNL control number ERID 0456.

Persons of ordinary skill in the art will recognize that many modifications may be made to the present invention without departing from the spirit and scope of the present invention. The embodiment described herein is meant to be illustrative only and should not be taken as limiting the invention, which is defined in the following claims.

We claim:

1. Mesogens having the following general formula: 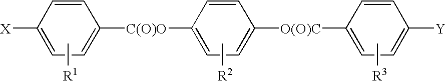
wherein
X and Y independently are selected from the group consisting of amino groups, polymerizable groups, and groups having the following general structure: 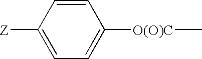
wherein Z is selected from the group consisting of terminal functionalities and polymerizable groups; provided that when X is a polymerizable group, Y is an amino group;
R² is a bulky organic group having a bulk greater than R¹ and R³ whereby, when both X and Y are polymerizable groups, said bulk is adapted to provide sufficient steric hindrance to achieve a nematic state at room temperature while suppressing crystallinity at room temperature, thereby providing effective rheology and workability at room temperature; and
R¹ and R³ are selected from groups less bulky than R² adapted to maintain said nematic state.

2. The mesogens of claim 1 wherein said polymerizable groups are selected from the group comprising a polymerizable unsaturated carbon-carbon bond.

3. The mesogens of claim 1 wherein said polymerizable groups are selected from the group consisting of acryloyloxy alkoxy groups and methacryloyloxy alkoxy groups comprising an alkyl moiety having from 2 to 12 carbon atoms.

4. The mesogens of claim 3 wherein said alkyl moiety is selected from the group consisting of alkyl groups consisting of CH₂ groups and alkyl groups wherein one or more of said CH₂ groups comprises a substitute group selected from the group consisting of oxygen, sulfur, and an ester group; provided that at least 2 carbon atoms separate said oxygen from said ester group.

5. The mesogens of claim 3 wherein said alkyl moiety consists essentially of a total of from 2 to 9 groups selected from the group consisting of said CH₂ groups and said substitute groups.

6. The mesogens of claim 3 wherein said alkyl moiety consists essentially of a total of from 2 to 6 groups selected from the group consisting of said CH₂ groups and said substitute groups.

7. The mesogens of claim 1 wherein R² is selected from the group consisting of alkyl groups having from about 1 to 6 carbon atoms and aryl groups.

8. The mesogens of claims 1 wherein R² is selected from the group consisting of methyl groups, t-butyl groups, isopropyl groups, secondary butyl groups, and phenyl groups, preferably selected from the group consisting of a methyl group and a t-butyl group.

9. The mesogens of claims 3 wherein R² is selected from the group consisting of methyl groups, t-butyl groups, isopropyl groups, secondary butyl groups, and phenyl groups, preferably selected from the group consisting of a methyl group and a t-butyl group.

10. The mesogens of claim 8 wherein R and R³ are selected from the group consisting of hydrogen and a methyl group.

11. The mesogens of any of claim 1 wherein said terminal functionalities comprise spacer groups.

12. The mesogens of claim 1 wherein said terminal functionalities independently are selected from the group consisting of hydroxyl groups, amino groups, sulfhydryl groups.

13. Mesogens having the following general formula: 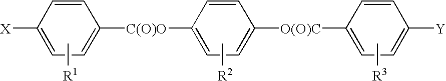
wherein
X and Y independently are selected from the group consisting of amino groups, polymerizable groups; provided that when X is a polymerizable group, Y is an amino group;
R² is a bulky organic group having a bulk greater than R¹ and R³ whereby, when both X and Y are polymerizable groups, said bulk is adapted to provide sufficient steric hindrance to achieve a nematic state at room temperature while suppressing crystallinity at room temperature, thereby providing effective rheology and workability at room temperature; and
R¹ and R³ are selected from groups less bulky than R² adapted to maintain said nematic state.

14. The mesogens of claim 13 wherein said polymerizable groups are selected from the group comprising a polymerizable unsaturated carbon-carbon bond.

15. The mesogens of claim 1 wherein said polymerizable groups are selected from the group consisting of acryloyloxy alkoxy groups and methacryloyloxy alkoxy groups comprising an alkyl moiety having from 2 to12 carbon atoms.

16. The mesogens of claim 15 wherein said alkyl moiety is selected from the group consisting of alkyl groups consisting of CH₂ groups and alkyl groups wherein one or more of said CH₂ groups comprises a substitute group selected from the group consisting of oxygen, sulfur, and an ester group; provided that at least 2 carbon atoms separate said oxygen from said ester group.

17. The mesogens of claim 16 wherein said alkyl moiety consists essentially of a total of from 2 to 9 groups selected from the group consisting of said CH₂ groups and said substitute groups.

18. The mesogens of claim 16 wherein said alkyl moiety consists essentially of a total of from 2 to 6 groups selected from the group consisting of said CH₂ groups and said substitute groups.

19. The mesogens of claim 13 wherein R² is selected from the group consisting of alkyl groups having from about 1 to 6 carbon atoms and aryl groups.

20. The mesogens of claims 13 wherein R² is selected from the group consisting of methyl groups, t-butyl groups, isopropyl groups, secondary butyl groups, and phenyl groups, preferably selected from the group consisting of a methyl group and a t-butyl group.

21. The mesogens of claims 15 wherein R² is selected from the group consisting of methyl groups, t-butyl groups, isopropyl groups, secondary butyl groups, and phenyl groups, preferably selected from the group consisting of a methyl group and a t-butyl group.

22. The mesogens of claim 20 wherein R and R³ are selected from the group consisting of hydrogen and a methyl group.

23. The mesogens of claim 21 wherein R and R³ are selected from the group consisting of hydrogen and a methyl group.

24. The mesogens of any of claim 13 wherein at least one of X or Y comprises a spacer group.

25. The mesogens of any of claim 23 wherein at least one of X or Y comprises a spacer group.

26. The mesogens of claim 13 wherein at least one of X or Y is selected from the group consisting of cinnamoyloxy groups.

27. The mesogens of claim 23 wherein at least one of X or Y is selected from the group consisting of cinnamoyloxy groups.

28. Mesogens having the following general formula: 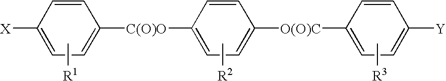
wherein X and Y independently are selected from the group consisting of terminal functionalities and polymerizable groups, at least one of X and Y having the following general structure: 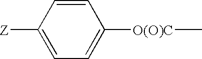
wherein Z is selected from the group consisting of terminal functionalities and polymerizable groups;
R² is a bulky organic group having a bulk greater than R¹ and R³ whereby, when both X and Y are polymerizable groups, said bulk is adapted to provide sufficient steric hindrance to achieve a nematic state at room temperature while suppressing crystallinity at room temperature, thereby providing effective rheology and workability at room temperature; and
R¹ and R³ are selected from groups less bulky than R² adapted to maintain said nematic state.

29. The mesogens of claim 28 wherein said terminal functionalities independently are selected from the group consisting of hydroxyl groups, amino groups, sulfhydryl groups, and spacer groups.

30. The mesogens of claim 28 wherein said terminal functionalities comprise spacer groups.

31. The mesogens of claim 29 wherein said terminal functionalities comprise spacer groups.

32. The mesogens of claim 28 wherein said polymerizable groups are selected from the group comprising a polymerizable unsaturated carbon-carbon bond.

33. The mesogens of claim 29 wherein said polymerizable groups are selected from the group comprising a polymerizable unsaturated carbon-carbon bond.

34. The mesogens of claim 30 wherein said polymerizable groups are selected from the group comprising a polymerizable unsaturated carbon-carbon bond.

35. The mesogens of claim 31 wherein said polymerizable groups are selected from the group comprising a polymerizable unsaturated carbon-carbon bond.

36. The mesogens of claim 28 wherein said polymerizable groups are selected from the group consisting of acryloyloxy alkoxy groups and methacryloyloxy alkoxy groups comprising an alkyl moiety having from 2 to12 carbon atoms.

37. The mesogens of claim 29 wherein said polymerizable groups are selected from the group consisting of acryloyloxy alkoxy groups and methacryloyloxy alkoxy groups comprising an alkyl moiety having from 2 to 12 carbon atoms.

38. The mesogens of claim 30 wherein said polymerizable groups are selected from the group consisting of acryloyloxy alkoxy groups and methacryloyloxy alkoxy groups comprising an alkyl moiety having from 2 to 12 carbon atoms.

39. The mesogens of claim 31 wherein said polymerizable groups are selected from the group consisting of acryloyloxy alkoxy groups and methacryloyloxy alkoxy groups comprising an alkyl moiety having from 2 to 12 carbon atoms.

40. The mesogens of claim 36 wherein said alkyl moiety is selected from the group consisting of alkyl groups consisting of CH₂ groups and alkyl groups wherein one or more of said CH₂ groups comprises a substitute group selected from the group consisting of oxygen, sulfur, and an ester group; provided that at least 2 carbon atoms separate said oxygen from said ester group.

41. The mesogens of claim 37 wherein said alkyl moiety is selected from the group consisting of alkyl groups consisting of CH₂ groups and alkyl groups wherein one or more of said CH₂ groups comprises a substitute group selected from the group consisting of oxygen, sulfur, and an ester group; provided that at least 2 carbon atoms separate said oxygen from said ester group.

42. The mesogens of claim 38 wherein said alkyl moiety is selected from the group consisting of alkyl groups consisting of CH₂ groups and alkyl groups wherein one or more of said CH₂ groups comprises a substitute group selected from the group consisting of oxygen, sulfur, and an ester group; provided that at least 2 carbon atoms separate said oxygen from said ester group.

43. The mesogens of claim 39 wherein said alkyl moiety is selected from the group consisting of alkyl groups consisting of CH₂ groups and alkyl groups wherein one or more of said CH₂ groups comprises a substitute group selected from the group consisting of oxygen, sulfur, and an ester group; provided that at least 2 carbon atoms separate said oxygen from said ester group.

44. The mesogens of claim 43 wherein said alkyl moiety consists essentially of a total of from 2 to 9 groups selected from the group consisting of said CH₂ groups and said substitute groups.

45. The mesogens of claim 43 wherein said alkyl moiety consists essentially of a total of from 2 to 6 groups selected from the group consisting of said CH₂ groups and said substitute groups.

46. The mesogens of claim 26 wherein R² is selected from the group consisting of alkyl groups having from about 1 to 6 carbon atoms and aryl groups.

47. The mesogens of claim 35 wherein R² is selected from the group consisting of alkyl groups having from about 1 to 6 carbon atoms and aryl groups.

48. The mesogens of claim 39 wherein R² is selected from the group consisting of alkyl groups having from about 1 to 6 carbon atoms and aryl groups.

49. The mesogens of claim 43 wherein R² is selected from the group consisting of alkyl groups having from about 1 to 6 carbon atoms and aryl groups.

50. The mesogens of claim 45 wherein R² is selected from the group consisting of alkyl groups having from about 1 to 6 carbon atoms and aryl groups.

51. The mesogens of claims 26 wherein R² is selected from the group consisting of methyl groups, t-butyl groups, isopropyl groups, secondary butyl groups, and phenyl groups, preferably selected from the group consisting of a methyl group and a t-butyl group.

52. The mesogens of claims 35 wherein R² is selected from the group consisting of methyl groups, t-butyl groups, isopropyl groups, secondary butyl groups, and phenyl groups, preferably selected from the group consisting of a methyl group and a t-butyl group.

53. The mesogens of claims 39 wherein R² is selected from the group consisting of methyl groups, t-butyl groups, isopropyl groups, secondary butyl groups, and phenyl groups, preferably selected from the group consisting of a methyl group and a t-butyl group.

54. The mesogens of claims 45 wherein R² is selected from the group consisting of methyl groups, t-butyl groups, isopropyl groups, secondary butyl groups, and phenyl groups, preferably selected from the group consisting of a methyl group and a t-butyl group.

55. The mesogens of claims 43 wherein R² is selected from the group consisting of methyl groups, t-butyl groups, isopropyl groups, secondary butyl groups, and phenyl groups, preferably selected from the group consisting of a methyl group and a t-butyl group.

56. The mesogens of claim 46 wherein R and R³ are selected from the group consisting of hydrogen and a methyl group.

57. The mesogens of claim 50 wherein R and R³ are selected from the group consisting of hydrogen and a methyl group.

58. The mesogens of claim 51 wherein R and R³ are selected from the group consisting of hydrogen and a methyl group.

59. The mesogens of claim 55 wherein R and R³ are selected from the group consisting of hydrogen and a methyl group.

60. The mesogens of claim 26 wherein at least one of X or Y is selected from the group consisting of cinnamoyloxy groups.

61. The mesogens of claim 55 wherein at least one of X or Y is selected from the group consisting of cinnamoyloxy groups.

62. The mesogens of claim 59 wherein at least one of X or Y is selected from the group consisting of cinnamoyloxy groups.
